(19) United States
(12) Patent Application Publication
(10) Pub. No.: US 2008/0221049 A1
(43) Pub. Date: Sep. 11, 2008

(54) Free-flowing lyophilized tobramycin formulation

(75) Inventors: K. Keith Kwok, Long Grove, IL (US); Kang Yong Yang, Flossmoor, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: APP Pharmaceuticals, LLC., Schaumburg, IL (US)

(21) Appl. No.: 12/080,665

(22) Filed: Apr. 4, 2008

Related U.S. Application Data
(63) Continuation of application No. 10/827,024, filed on Apr. 19, 2004, now abandoned.

(51) Int. Cl. A61K 31/7036

(52) U.S. Cl. 514/40

(57) ABSTRACT

The invention provides among other things a stable, sterile pharmaceutical formulation comprising lyophilized tobramycin, wherein the lyophilized tobramycin is in the form of a free-flowing powder. The invention also provides a method of producing a stable, sterile pharmaceutical product comprising lyophilized tobramycin. The invention also provides a pharmaceutical dosage form comprising the pharmaceutical formulation, as well as a method of treating a disease in a patient comprising administering a solution of the pharmaceutical formulation to a patient.

### CROSS-REFERENCE TO RELATED APPLICATIONS

This application is a continuation of U.S. patent application Ser. No. 10/827,024 filed Apr. 19, 2004.

### FIELD OF THE INVENTION

This invention pertains among other things to lyophilized tobramycin, solutions thereof, and methods of preparing and using lyophilized tobramycin.

### BACKGROUND OF THE INVENTION

Tobramycin sulfate is an aminoglycoside antibiotic derived from Streptomyces tenebrarius which has the formula:

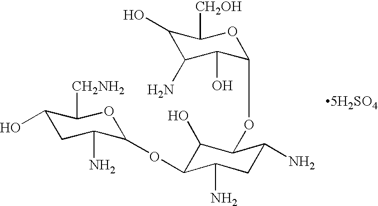

Tobramycin sulfate is chemically described as 0-3-amino-3-deoxy-α-D-glucopyranosyl-(1→4) —O—[2,6-diamino-2,3,6-trideoxy-α-D-ribo-hexopyranosyl-(1→6)]-2-deoxy-L-streptamine, sulfate (2:5)(salt). Tobramycin sulfate has the molecular formula (C₁₈H₃₇N₅O₉)₂.5H₂SO₄, and has a molecular weight of 1425.45.

Tobramycin sulfate is approved in the United States for the treatment of the following infections when caused by susceptible organisms: septicemia, complicated and recurrent urinary tract infections, lower respiratory infections, serious skin and soft tissue infections including burns and peritonitis, and central nervous system (CNS) infections caused by organisms resistant to other antibiotics. Tobramycin sulfate is typically active against most strains of the following organisms in vitro and in clinical infections: P. aeruginosa, Proteus sp. (indole-positive and indole-negative), including P. mirabilis, M. morganii, P. rettgeri, and P. vulgaris, E. coli, Klebsiella-Enterobacter-Serratia group, Citrobacter sp., Providencia sp., Staphylococci, including S. aureus (coagulase-positive and coagulase-negative).

Tobramycin sulfate is currently sold in the U.S. as a sterile dry powder in 1.2 g Pharmacy Bulk Package dose vials (e.g., marketed by Eli Lilly and Company under the tradename Nebcin®). For therapeutic applications, the powder is dissolved in a sterile aqueous vehicle such as Sterile Water for Injection, USP, and is administered by injection. Typically, the 1.2 g dosage form is constituted in 30 mL of the aqueous vehicle to achieve a final concentration of 40 mg/mL. Solutions of tobramycin sulfate may be diluted further in injectable fluids such as 5% Dextrose in water, USP, and 0.9% Sodium Chloride Injection, USP. Optionally, the tobramycin formulation is first constituted (e.g., with sterile water) and then further diluted (e.g., with a sodium chloride solution).

Tobramycin sulfate is normally administered via intramuscular (i.m.) or intravenous (i.v.) injection at a dosage between about 2 mg/kg per day to about 5 mg/kg per day for adults, depending on the infection to be treated. Tobramycin sulfate is normally administered to children at a dosage of 6 to 7.5 mg/kg per day, and is administered to neonates at a dosage up to 4 mg/kg per day. The duration of treatment typically is seven to ten days. After reconstitution, e.g., with 30 mL of Sterile Water for Injection, tobramycin solutions should be used within 24 hours if stored at room temperature, or within 96 hours if stored under refrigeration.

The manufacture of tobramycin sterile powder involves a powder fill operation that presents a number of practical problems. During the production of the sterile powder, the powder is processed in a way that causes variations in the flow properties. The variation in flow properties greatly impairs the accuracy of dosage during the filling process. The sterile powder fill operation further employs specialized equipment. The powder fill operation is costly and may be further complicated by the risk of microbial contamination during the operation. Moreover, the powder fill process creates risks of accidental exposure to the powder by personnel that are involved in the production process.

Approaches for manufacturing lyophilized forms of tobramycin sulfate have been proposed including tobramycin for injection marketed by Pharma-Tek Inc. While this lyophilization process overcomes some of the problems associated with the crystalline powder, it produces a cake in which the lyophilizate particles adhere to each other and to the walls of its container.

A method for freeze drying tobramycin sulfate to produce a friable freeze dried powder has been developed that entails use of a tert-butanol/water cosolvent system (Nail et al., J. Pharm. Sciences, 91:1147-1155 (2002). While this method produces a loose powder that “readily breaks apart”, the method disclosed is not adapted for the commercial production of tobramycin sulfate.

Thus, there remains a need for a stable, sterile form of lyophilized tobramycin having a smooth, uniform cake and non-adherent particles, methods of producing such a formulation that are appropriate for commercial scale, and methods of using such a formulation. The invention provides such a formulation and methods. These and other advantages of the present invention, as well as additional inventive features, will be apparent from the description of the invention provided herein.

### BRIEF SUMMARY OF THE INVENTION

The invention provides among other things a stable, sterile pharmaceutical formulation comprising lyophilized tobramycin, wherein the lyophilized tobramycin is in the form of a free-flowing powder. The invention also provides a solution prepared by dissolving the pharmaceutical formulation in an aqueous vehicle. The invention further provides a liquid composition comprising tobramycin (e.g., tobramycin sulfate) and a solvent which comprises tert-butyl alcohol.

The invention also provides a method of producing a stable, sterile pharmaceutical product comprising lyophilized tobramycin, which method comprises preparing a composition comprising tobramycin (e.g., tobramycin sulfate) and a solvent, which solvent comprises tert-butyl alcohol, and lyophilizing the composition, wherein the lyophilized tobramycin is in the form of a free-flowing powder.

The invention further provides a method of producing a stable, sterile pharmaceutical formulation comprising lyophilized tobramycin, which method comprises (a) preparing a liquid composition comprising tobramycin (e.g., tobramycin sulfate) and a solvent which comprises tert-butyl alcohol, (b) freezing the composition to a temperature of from about −10° C. to about −70° C., to produce a frozen mixture, wherein the temperature is maintained for from at least about 1 hour to about 30 hours, (c) subjecting the frozen mixture to a primary drying stage, which comprises applying a vacuum to reduce the pressure by an amount effective to remove aqueous solvent from the frozen mixture, and, while applying the vacuum, changing the temperature of the frozen mixture to a primary drying temperature, wherein the primary drying temperature is from about −15° C. to about 20° C., and wherein the primary drying temperature is maintained for at least about 40 hours to about 80 hours, to produce a first intermediate, and (d) subjecting the first intermediate to a secondary drying stage, which comprises applying a vacuum to reduce the pressure by an amount effective to remove aqueous solvent from the first intermediate, and, while applying the vacuum, changing the temperature of the frozen mixture to a secondary drying temperature, wherein the secondary drying temperature is from about 30° C. to about 45° C., and wherein the secondary drying temperature is maintained for at least about 15 hours to about 30 hours.

The invention additionally provides a pharmaceutical dosage form comprising a sealed container and a pharmaceutical formulation comprising a therapeutically effective amount of lyophilized tobramycin contained within the container. The invention provides a method of treating a disease in a patient, which comprises dissolving the above-described pharmaceutical formulation in a pharmaceutically acceptable solvent to produce a pharmaceutically acceptable solution, and administering the solution to a patient in need thereof.

### DETAILED DESCRIPTION OF THE INVENTION

The invention provides among other things a stable, sterile pharmaceutical formulation comprising lyophilized tobramycin, wherein the lyophilized tobramycin is in the form of a free-flowing powder. The lyophilized tobramycin of the present invention is a white to off-white solid of high purity. The lyophilized tobramycin of the present invention preferably has a purity of greater than about 90% (i.e., contains about 10% or less of total impurities based on the total weight of tobramycin), more preferably has a purity of about 96% or greater (i.e., contains about 4% or less of total impurities based on the total weight of tobramycin), and even more preferably has a purity of about 98% or greater (i.e., contains about 2% or less of total impurities based on the total weight of tobramycin). Most preferably, the lyophilized tobramycin has a purity of about 98%, about 98.5%, or about 99% (i.e., contains about 2%, about 1.5%, or about 1.0%, respectively, of total impurities based on the total weight of tobramycin). Purity can be assessed based on potency. Purity also can be determined by high performance liquid chromatography assay (e.g., allowing separation of pure lyophilized tobramycin from impurities, and quantitation of the relative amounts by the determination of the peak area of pure tobramycin and/or the impurities area as compared to the total peak area), or by a similar method.

The lyophilized tobramycin formulation can comprise any suitable amount of tobramycin, but preferably comprises a therapeutically effective amount of tobramycin. A “therapeutically effective amount” means an amount sufficient to show a meaningful benefit in an individual, e.g., promoting at least one aspect of antimicrobial activity, or treatment, healing, prevention, or amelioration of other relevant medical condition(s) such as that associated with a particular microbial infection. Therapeutically effective amounts may vary depending upon the biological effect desired in the individual, condition to be treated, and the individual. In this regard, the lyophilized tobramycin preferably is present in the formulation in an amount from about 0.5 grams to about 5.0 grams (e.g., about 1.0 g, about 1.5 g, about 2.0 g, about 2.5 g, about 3.0 g, about 3.5 g, about 4.0 g, about 4.5 g, or about 5.0 g). Most preferably, the lyophilized tobramycin is present in an amount of from about 0.5 g to about 1.5 g, especially in an amount of about 1.2 g.

The inventive lyophilized tobramycin preferably is in the form of a free-flowing powder. The term “free-flowing powder,” as used herein, refers to a powder that shows minimal or no compaction, and which contains particles that adhere minimally or not at all to each other or to surfaces with which the particles are in contact (e.g., container walls). In the event that minimal compaction or adherence is observed, the powder according to the invention is such that the minimally compacted or adhered powder readily breaks up into a free-flowing form with gentle tapping or shaking of the vessel containing the powder. The flowability of a powder is affected by a variety of factors, including physical characteristics of the particles themselves (e.g., size, shape, angularity, and hardness), as well as external factors such as humidity, vibration, and aeration. The flow properties (also referred to as rheological properties) of a powder can be measured using any suitable technique known in the art including, for example, measuring the angle of repose of the powder and timing the flow of a powder through an aperture. Alternatively, a powder rheometer can be used to determine flowability.

The lyophilized tobramycin formulation can be prepared using any suitable form of tobramycin, including in particular tobramycin base (i.e., 0-3-amino-3-deoxy-α-D-glucopyranosyl-(1->6)-O-(2,6-diamino-2,3,6-trideoxy-α-D-ribo-hexopyranosyl-(1->4)-2-deoxy-d-streptamine)) and tobramycin salts, especially tobramycin sulfate. Optionally, rather than a tobramycin sulfate salt, another tobramycin salt can be used instead in the formulations of the invention, e.g., a sugar acid salt (e.g., as described in U.S. Pat. Nos. 5,595,977 and 6,077,822), a nitrate salt (e.g., as described in U.S. Patent Application 2003/0105066), and the sparingly-soluble tobramycin salts tobramycin laurate, tobramycin myristate, and tobramycin dodecyl sulfate (e.g., as described in U.S. Patent Application 2004/0048786). A preferred form of tobramycin according to the invention for preparation of the lyophilized tobramycin formulation is tobramycin sulfate.

In a preferred embodiment of the invention, the lyophilized tobramycin formulation has a low moisture content. The moisture content of the inventive lyophilized tobramycin is the result of residual aqueous solvent (i.e., water) that remains in the formulation after the lyophilization process. Additionally, some small amount of organic solvent optionally remains in the formulation following lyophilization. The organic solvent content and/or the moisture content can be the product of any suitable solvent that is used in the method of producing the lyophilized tobramycin formulation described herein. Suitable solvents include, for example, aqueous solvents (i.e., water, which could contribute to the moisture content), organic solvents (which could contribute to the organic solvent content), or a combination of an aqueous solvent and an organic solvent (which could contribute to both the moisture content and the organic solvent content). Preferably, the solvent according to the invention comprises water and an organic solvent. Suitable organic solvents include, for example, alcohols (e.g., ethanol, isopropyl alcohol, and tert-butyl alcohol (TBA)). More preferably, the organic solvent is a sterically hindered alcohol, such as tert-butyl alcohol; however, any suitable organic solvent can be used in the invention. Most preferably, the organic solvent is tert-butyl alcohol. In a preferred embodiment, the solvent according to the invention preferably comprises tert-butyl alcohol and water. The moisture content of the inventive lyophilized tobramycin formulation desirably is measured as the content of water present in the lyophilized tobramycin formulation, whereas the organic solvent content of the inventive lyophilized tobramycin formulation desirably is measured as the content of organic solvent present in the lyophilized tobramycin formulation. In this regard, the lyophilized tobramycin preferably contains less than about 2 wt % of water (where the wt % is the % water relative to the total weight of the lyophilized tobramycin formulation). The lyophilized tobramycin preferably contains less than about 1.1 wt % of organic solvent such as tert-butyl alcohol (where the wt % is the % organic solvent relative to the total weight of the lyophilized tobramycin formulation.). More preferably, the lyophilized tobramycin sulfate contains about 1 wt %, about 0.9 wt %, about 0.8 wt %, or about 0.7 wt % of an organic solvent such as tert-butyl alcohol. When an organic solvent other than tert-butyl alcohol is employed, the organic solvent wt % can be adjusted accordingly, e.g., based on safety and/or toxicity concerns.

The components of the lyophilized tobramycin formulation of the invention (e.g., the tobramycin and the organic solvent) are further described below in the context of the liquid composition. The “liquid composition” is the formulation of the invention prior to lyophilization (e.g., the pre-lyophilization solution). While the pre-lyophilization solution may not be optimal for a pharmaceutical formulation, in some instances, however, it may be desirable to constitute (and optionally further dilute) a pharmaceutical lyophilized tobramycin formulation for use as a liquid (e.g., solution). Along these lines, a pharmaceutical formulation according to the invention comprises tobramycin (e.g., tobramycin sulfate) and solvent (as well as further optional components) preferably either in lyophilized form, or as a liquid. For the liquid compositions of the invention to serve as pharmaceutical tobramycin formulations, optionally additional components are included, such as preservatives (e.g., methylparaben and propylparaben), stabilizers (e.g., sugars), and buffering agents (e.g., sodium citrate, sodium hydroxide, sodium acetate, and others).

The inventive lyophilized tobramycin formulation can be contained within a sealed container. Preferably, the inventive lyophilized tobramycin formulation is contained within a container that is sealed aseptically. More preferably, the container is provided with an opening and a means for aseptically sealing the opening, e.g., such that the sealed container is fluidly sealed or the sealed opening is substantially impermeable to atmospheric gasses, moisture, pathogenic microorganisms, or the like. The container can be constructed of any suitable material such as, for example, glass, polypropylene, Dalkyo Resin CZ (sold by Dalkyo Gomu Seiko, Ltd.), polyethylene terephthalate, and the like. In a preferred embodiment, the container is constructed of glass. Suitable glass containers include, but are not limited to, glass vials. Suitable glass vials include molded and tubing glass vials such as, for example, Type I molded glass vials, and the like. Such molded and tubing glass vials can be obtained from Kimble Glass, Inc., Vineland, N.J., Wheaton Science Products, Millville, N.J., or other companies.

A suitable means for sealing the container can include, for example, a stopper, a cap, a lid, a closure, a covering which fluidly seals the container, or the like. Examples of suitable closures include closures that are suitable for medical vials, such as those described in U.S. Pat. No. 4,671,331, and references cited therein. The means for sealing the container are not limited to separate closures or closure devices. In a preferred embodiment, the means for aseptically sealing the container includes a stopper such as, for example, a stopper that is configured to fluidly seal the opening. Suitable stoppers include conventional medical grade stoppers which do not degrade or release significant amounts of impurities upon exposure to the constituted aqueous tobramycin solution. Preferably, the stopper is constructed of an elastomer, which is more preferably an elastomer that is pierceable by a hypodermic needle or a blunt cannula. Exemplary stoppers include 6720 GC gray rubber stoppers from American Stelmi Corporation, 4432/50 gray rubber stoppers from West Company, and the like.

Optionally, an outer seal is provided which covers and entirely surrounds the stopper. The outer seal can be constructed of any suitable material. When an outer seal is used, it is preferably fitted with a lid that can be easily manually removed to provide access to the stopper. Suitable outer seals can include, for example, Flip-off Aluminum/Polypropylene Seals (lacquered or non-lacquered aluminum), marketed by The West Company, Inc., and other manufacturers. Such seals include an outer rim made of a suitable material, such as aluminum, that entirely surrounds the lateral edge of the stopper and further include a lid (typically polypropylene or other suitable material) that entirely covers the upper surface of the stopper. The polypropylene lid can be “flipped” off e.g., by exerting upward pressure with a finger or thumb, to provide access to the stopper, e.g., so that it can be punctured with a hypodermic needle to deliver an aqueous vehicle for constitution (see, e.g., U.S. Pat. No. 6,136,814). Optionally, the seal can be removed in its entirety to allow the powder to be poured from the vial.

Preferably, the container contains a therapeutically effective dose of the inventive lyophilized tobramycin formulation and is of sufficient volume (i.e., has sufficient capacity) to contain the volume of solution that is recommended for constitution. More preferably, the container contains tobramycin in an amount which is an approved dosage for treating microbial infections, such as those described herein, and is of sufficient volume (i.e., has sufficient capacity) to contain the total volume of solution recommended for constitution. In a particularly preferred embodiment, the container volume is about 50 mL, and about 1.2 g of the inventive lyophilized tobramycin formulation are contained within the container.

The invention further provides a solution prepared by dissolving the inventive lyophilized tobramycin formulation in an aqueous vehicle. The aqueous vehicle is preferably a sterile aqueous vehicle that is normally used as liquid vehicle for injection. Suitable aqueous vehicles include, for example, sterile water (e.g., Sterile Water for Injection, USP), sodium chloride solutions (e.g., 0.9% Sodium Chloride Injection, USP), dextrose solutions (e.g., 5% or 10% Dextrose for Injection), sodium chloride/dextrose mixtures (e.g., 5% Dextrose and 0.225% Sodium Chloride for Injection, 5% Dextrose and 0.45% Sodium Chloride for Injection), Lactated Ringer's for Injection, and mixtures thereof. Optionally, the lyophilized tobramycin formulation is first reconstituted (e.g., with sterile water) and then further diluted (e.g., with a sodium chloride solution).

The inventive lyophilized tobramycin formulation can be dissolved in any suitable volume of the aqueous vehicle. Preferably, the lyophilized tobramycin formulation is dissolved in about 50 mL or less of vehicle (e.g., about 40 mL, about 30 mL, about 20 mL, about 10 mL, or about 5 mL). Preferably, the lyophilized tobramycin formulation is dissolved in the aqueous vehicle, such that the final concentration of tobramycin in the solution is about 40 mg/mL or less (e.g., is about 40 mg/mL, about 35 mg/mL, about 30 mg/mL, about 25 mg/mL, about 20 mg/mL, about 15 mg/mL, or 10 mg/mL, about 5 mg/mL). Preferably, after further dilution the final concentration of tobramycin in the solution is about 2 mg/mL or less (e.g., about 1.5 mg/mL, about 1.0 mg/mL, about 0.5 mg/mL, about 0.4 mg/mL, about 0.3 mg/mL, or about 0.2 mg/mL). Most preferably, the final concentration of tobramycin in the solution is from about 0.2 mg/mL to about 2 mg/mL.

As described above, the invention also provides a liquid composition comprising tobramycin (e.g., tobramycin sulfate) and a solvent, which solvent comprises tert-butyl alcohol. The liquid composition can comprise any suitable amount of tert-butyl alcohol, so long as, upon lyophilization of the liquid composition, the tobramycin is in the form of a free-flowing powder. In this regard, the liquid composition preferably contains from about 1.5% to about 9.0% by volume (v/v) of tert-butyl alcohol (e.g., about 9.0% v/v, about 8.5% v/v, about 8.0% v/v, about 7.5% v/v, about 6.5% v/v, about 5.5% v/v, about 4.5% v/v, about 3.5% v/v, about 2.5% v/v, or about 1.5% v/v). Most preferably, the liquid composition comprises about 4.5% by volume or less (e.g., about 4% v/v, about 3.5% v/v, about 3% v/v, about 2% v/v, about 1% v/v, or about 0.5% v/v) of tert-butyl alcohol.

The liquid composition can comprise any suitable amount of tobramycin. In a preferred embodiment of the invention, the tobramycin (e.g., tobramycin sulfate) is present in the liquid composition in an amount of from about 60 mg/mL to about 100 mg/mL. In a particularly preferred embodiment of the invention, the tobramycin (e.g., tobramycin sulfate) is present in the liquid composition in an amount of from about 70 mg/mL to about 90 mg/mL (e.g., about 70 mg/mL, about 75 mg/mL, about 80 mg/mL, about 85 mg/mL, or about 90 mg/mL). Most preferably, the tobramycin (e.g., tobramycin sulfate) in the liquid composition is present in an amount of about 83.2 mg/mL. One of ordinary skill in the art would know how to modify the composition for use of forms other than tobramycin sulfate.

In addition, the solvent of the liquid composition preferably contains sterile water, such as Sterile Water for Injection, USP.

The inventive liquid composition can further comprise excipients that are routinely employed in lyophilization formulations. Such excipients include, for example, buffering agents, surfactants, cryoprotectants, and bulking agents. Mannitol, for example, typically is used in the art as an excipient in lyophilization formulations. However, other suitable excipients can be included which preferably do not deleteriously impact the properties of the inventive liquid composition. Examples of such excipients (e.g., buffering agents) include sodium or potassium phosphate, citric acid, lactic acid, tartaric acid, gelatin, glycine, and carbohydrates such as lactose, maltose, dextrose, dextran, hetastarch, and the like. The excipients can be used alone or in combination to provide a cake of good quality which readily disperses in an aqueous vehicle upon reconstitution.

The invention provides a method of producing a stable, sterile pharmaceutical product comprising lyophilized tobramycin, which method comprises preparing a composition comprising tobramycin (e.g., tobramycin sulfate) and a solvent, which solvent comprises tert-butyl alcohol, and lyophilizing the composition, wherein the lyophilized tobramycin is in the form of a free-flowing powder.

The invention also provides a method for producing the inventive lyophilized tobramycin formulation, which comprises (a) preparing a composition comprising tobramycin (e.g., tobramycin sulfate) and a solvent which comprises tert-butyl alcohol, (b) freezing the composition to a temperature of from about −10° C. to about −70° C., to produce a frozen mixture, (c) subjecting the frozen mixture to a primary drying stage, which comprises applying a vacuum to reduce the pressure by an amount effective to remove the solvent from the frozen mixture, and, while applying the vacuum, changing (e.g., raising) the temperature to a primary drying temperature, wherein the primary drying temperature is from about −15° C. to about 20° C., to produce a first intermediate, and (d) subjecting the first intermediate to a secondary drying stage, which comprises applying a vacuum to reduce the pressure by an amount effective to remove the solvent from the first intermediate, and, while applying the vacuum, changing (e.g., raising) the temperature to a secondary drying temperature, wherein the secondary drying temperature is from about 30° C. to about 45° C., to produce the pharmaceutical formulation. Descriptions of the tobramycin (e.g., tobramycin sulfate) and the solvent comprising tert-butyl alcohol, and components thereof, set forth above in connection with the inventive pharmaceutical formulation and liquid composition, also are applicable to those same aspects of the inventive method.

The composition is “frozen” or cooled to a temperature that freezes the tert-butyl alcohol solvent. Preferably, the liquid composition is frozen sufficiently to allow for its removal under reduced pressure (e.g., by sublimation). Desirably, the liquid composition is frozen to a temperature of about −10° C. or lower (e.g., from about −10° C. to about −70° C., from about −20° C. to about −70° C., from about −30° C. to about −70° C., or from about −30° C. to about −60° C.), and is preferably frozen to a temperature of about −30° C. or lower (e.g., from about −30° C. to about −50° C.). More preferably, the liquid composition is frozen to a temperature of about −45° C. or lower (e.g., from about −45° C. to about −60° C.). Most preferably, the liquid composition is frozen to a temperature of about −47° C., −48° C., −49° C., or −50° C.

The composition can be frozen rapidly (e.g., by contacting a container of the solution in a cooling bath), or by cooling in stages (e.g., by lowering the temperature incrementally at progressively lower temperatures until the frozen mixture is obtained). Alternatively, the sterile solution can be frozen by continuously cooling at a substantially constant rate until the frozen mixture is obtained. For example, the composition can be frozen by cooling at a substantially constant rate of about 5° C. per minute or less (e.g., from about 0.1 to about 5° C. per minute), at a rate of about 3° C. per minute or less (e.g., from about 0.1 to about 3° C. per minute), at a rate of about 2° C. per minute or less (e.g., from about 0.1 to about 2° C. per minute), or at a rate of about 1° C. per minute or less (e.g., from about 0.1 to about 1° C. per minute, or from about 0.1 to about 0.5° C. per minute), until the frozen mixture is obtained. Most preferably, the composition is frozen by cooling at a rate of about 0.1° C. per minute, about 0.2° C. per minute, about 0.3° C. per minute, about 0.4° C. per minute, or about 0.5° C. per minute. Alternatively, the composition can be frozen using a combination of incremental cooling stages and one or more continuous cooling cycles (e.g., continuously cooling at a substantially constant rate) until the frozen mixture is obtained.

After the frozen mixture is obtained, the temperature can be maintained for any suitable length of time. Preferably, the temperature at which the composition is frozen is maintained for at least about 1 hour to about 30 hours (e.g., about 1 hour, about 5 hours, about 15 hours, about 25 hours, or about 30 hours). More preferably, the temperature at which the composition is frozen is maintained for at least about 10 hours to about 20 hours (e.g., about 10 hours, about 12 hours, about 15 hours, or about 19 hours). Most preferably, the temperature at which the composition is frozen is maintained for at least about 15 hours.

The primary drying temperature in the primary drying phase is preferably from about −30° C. to about 35° C., especially from about −30° C. to about 20° C., more preferably from about −15° C. to about 20° C., desirably from about −15° C. to about 10° C., and still more desirably from about −15° C. to about 0° C. Most preferably, the primary drying temperature is from about −15° C. to about −10° C. (e.g., is about −15° C.).

In the primary drying stage, the temperature of the frozen composition can be changed in stages (e.g., raised or lowered incrementally at progressively higher or lower temperatures until the primary drying temperature is attained). Alternatively, the temperature of the frozen composition in the primary drying stage can be changed continuously (e.g., raised or lowered at a substantially constant rate) until the primary drying temperature is attained. Preferably, the temperature of the frozen composition in the primary drying stage is changed at a rate of about 5° C. per minute or less (e.g., from about 0.05° C. to about 2° C. per minute). More preferably, the temperature of the frozen composition in the primary drying stage is changed at a rate of about 3° C. per minute or less (e.g., from about 0.05° C. to about 3° C. per minute). Still more preferably, the temperature of the frozen composition in the primary drying stage is changed at a rate of about 2° C. per minute or less (e.g., from about 0.1° C. to about 2° C. per minute). Most preferably, the temperature of the frozen composition in the primary drying stage is changed at a rate of about 1° C. per minute or less (e.g., from about 0.1° C. to about 1° C. per minute, or from about 0.1° C. to about 0.5° C. per minute). In a particularly preferred embodiment, the temperature of the frozen composition in the primary drying stage is changed at a rate of about 0.5° C. per minute or less (e.g., about 0.5° C. per minute, about 0.4° C. per minute, about 0.3° C. per minute, about 0.2° C. per minute, or about 0.1° C. per minute).

The primary drying temperature in the primary drying stage is preferably maintained (e.g., held at a substantially constant temperature or kept within a particular range) until substantially all of the tert-butyl alcohol solvent is removed. In this regard, the primary drying temperature desirably is maintained for at least about 40 hours to about 80 hours (e.g., about 40 hours, about 50 hours, about 60 hours, about 70 hours, or about 80 hours). Preferably, the primary drying temperature is maintained for at least about 60 hours to 70 hours (e.g., about 62 hours, about 65 hours, or about 67 hours). Most preferably, the primary drying temperature is maintained for at least about 67 hours.

In general, as the temperature is raised during the primary drying stage, the internal temperature “lags” behind (i.e., is lower than) the external temperature (sometimes referred to as the “shelf temperature”). In some instances when the external temperature is raised during the primary drying stage, the internal temperature can lag behind the external temperature by as much as about 10° C., or even more. Typically, the removal of substantially all of the solvent can be determined by comparing the internal temperature with the external temperature. The temperature of the frozen mixture and the external temperature can be measured using any suitable means, e.g., a thermometer, a thermocouple, or the like. In most instances, substantially all of the tert-butyl alcohol solvent is removed when the internal temperature remains steady or is about equal to (e.g., is slightly less than, is equal to, or slightly exceeds) the external temperature. In a preferred embodiment, the primary drying temperature is maintained until the temperature of the frozen mixture is about equal to the primary drying temperature.

The secondary drying temperature in the secondary drying stage can range from about 0° C. to about 45° C., but is preferably from about 10° C. to about 40° C. More preferably, the secondary drying temperature is about ambient temperature (e.g., from about 20° C. to about 45° C., even more preferably from about 30° C. to about 45° C.), and is most preferably from about 20° C. to about 35° C. (e.g., is about 30° C.). In the secondary drying stage, the temperature of the frozen composition can be changed (e.g., raised or lowered) at a rate which is the same or different than the rate at which the temperature is changed in the primary drying stage. For example, the temperature of the frozen composition in the secondary drying stage can be changed in stages (e.g., raised or lowered incrementally at progressively higher or lower temperatures until the secondary drying temperature is attained).

Alternatively, the temperature of the frozen composition in the secondary drying stage can be changed continuously (e.g., at a substantially constant rate) until the secondary drying temperature is attained. Preferably, the temperature of the frozen composition in the secondary drying stage is changed at a rate of about 5° C. per minute or less (e.g., from about 0.1° C. to about 5° C. per minute). More preferably, the temperature of the frozen composition in the secondary drying stage is changed at a rate of about 3° C. per minute or less (e.g., from about 0.1° C. to about 3° C. per minute). Still more preferably, the temperature of the frozen composition in the secondary drying stage is changed at a rate of about 2° C. per minute or less (e.g., from about 0.1° C. to about 2° C. per minute). Most preferably, the temperature of the frozen composition in the secondary drying stage is changed at a rate of about 1° C. per minute or less (e.g., from about 0.1° C. to about 1° C. per minute, or from about 0.1° C. to about 0.5° C. per minute). In a particularly preferred embodiment, the temperature of the frozen composition in the secondary drying stage is changed at a rate of about 0.5° C. per minute or less (e.g., about 0.5° C. per minute, about 0.4° C. per minute, about 0.3° C. per minute, about 0.2° C. per minute, or about 0.1° C. per minute).

Preferably, the secondary drying temperature in the secondary drying stage is maintained until the moisture content is less than about 2.0 wt % (% liquid relative to the dry weight of the lyophilized tobramycin formulation). More preferably, the secondary drying temperature in the secondary drying stage is held until the moisture content is about 1.0 wt % or less. Most preferably, the secondary drying temperature in the secondary drying stage is held until the moisture content is about 0.5 wt % or less.

The secondary drying temperature preferably is maintained for at least about 5 hours, especially for from about 5 hours to about 30 hours (e.g., about 5 hours, about 15 hours, about 18 hours, about 20 hours, about 25 hours, or about 30 hours). More preferably, the secondary dying temperature is maintained for at least from about 10 hours to about 20 hours (e.g., about 10 hours, about 12 hours, about 15 hours, about 16 hours, about 17 hours, about 18 hours, or about 19 hours). Most preferably, the secondary drying temperature is maintained for about 15 hours, about 16 hours, about 17 hours, about 17.5 hours, about 18 hours, about 18.5 hours, or about 19 hours.

The primary drying stage is preferably carried out at a pressure of about 500 micron Hg (about 67 Pascal (Pa) or less (e.g., from about 10 micron Hg to about 500 micron Hg (or about 1 Pa to about 67 Pa)). More preferably, the pressure is about 300 micron Hg (about 40 Pa) or less (e.g., from about 10 micron Hg to about 300 micron Hg (or about 1 Pa to about 40 Pa)). Most preferably, the primary drying stage is carried out at a pressure of about 150 micron Hg (or about 20 Pa) or less (e.g., from about 10 micron Hg to about 150 micron Hg (or about 1 Pa to about 20 Pa)). In a particularly preferred embodiment, the primary drying stage is carried out at a pressure of about 100 micron Hg (about 13 Pa).

The secondary drying stage can be carried out at a pressure which is the same or different than the pressure at which the primary drying stage is carried out. Preferably, the secondary drying stage is carried out at a pressure of about 500 micron Hg (about 67 Pa) or less (e.g., from about 10 micron Hg to about 500 micron Hg (or about 1 Pa to about 67 Pa). More preferably, the pressure is about 300 micron Hg (or about 40 Pa) or less (e.g., from about 10 micron Hg to about 300 micron Hg (or about 1 Pa to about 40 Pa)). Most preferably, the primary drying stage is carried out at a pressure of about 150 micron Hg (or about 20 Pa) or less (e.g., from about 10 micron Hg to about 150 micron Hg (or about 1 Pa to about 20 Pa)). In a particularly preferred embodiment, the secondary drying stage is carried out at a pressure of about 100 micron Hg (or about 13 Pa).

The invention further provides a pharmaceutical dosage form comprising a sealed container and a pharmaceutical formulation comprising a therapeutically effective amount of lyophilized tobramycin contained within the container, wherein the lyophilized tobramycin is in the form of a free-flowing powder. Descriptions of the lyophilized tobramycin and free-flowing powder, and components thereof, set forth above in connection with the inventive pharmaceutical formulation, also are applicable to those same aspects of the inventive pharmaceutical dosage form. The pharmaceutical dosage form can be a sterile single-dose or sterile multiple-dose dosage form. Exemplary pharmaceutical dosage forms include a pharmaceutical dosage form comprising a sealed container (e.g., a container as described herein) and the inventive pharmaceutical formulation comprising a therapeutically effective amount of lyophilized tobramycin contained within the container.

The inventive pharmaceutical dosage form preferably includes a dose of lyophilized tobramycin of from about 0.5 grams to about 5.0 grams (e.g., about 1.0 g, about 1.5 g, about 2.0 g, about 2.5 g, about 3.0 g, about 3.5 g, about 4.0 g, about 4.5 g, or about 5.0 g) contained within the container. Most preferably, the pharmaceutical dosage form includes a dose of the lyophilized tobramycin of about 1.2 g contained within the container.

To prepare the pharmaceutical dosage form, the pharmaceutical formulation can be packaged in the container by any suitable method known in the art. In a preferred embodiment of the invention, the inventive pharmaceutical formulation is packaged in the container by a method comprising the steps of (a) filling one or more containers with a sterile liquid composition comprising a therapeutically effective amount of tobramycin (e.g. present as tobramycin sulfate) and a solvent comprising tert-butyl alcohol (e.g., a solvent comprising tert-butyl alcohol and water), each container defining an opening, (b) subjecting the composition in the one or more containers to the lyophilized tobramycin production method described herein, and (c) sealing the opening of the one or more containers, to produce the pharmaceutical dosage form.

The one or more containers preferably include one or more sterile vials, preferably glass vials, as described herein. The sealing step preferably includes sealing the opening using the means for aseptically sealing the opening described herein. The sealing means preferably includes a stopper as described herein.

The sterile liquid composition added to one or more containers (prior to lyophilization) preferably contains tobramycin in an amount of from about 60 mg/mL to about 100 mg/mL, and even more preferably contains tobramycin in an amount of from about 70 mg/mL to about 90 mg/mL (e.g., about 70 mg/mL, about 75 mg/mL, about 80 mg/mL, about 85 mg/mL, or about 90 mg/mL). In a particularly preferred embodiment, the concentration of the sterile solution is about 83.2 mg/mL and the one or more containers (which are most preferably vials) are filled with 50 mL or less (e.g., about 40 mL, about 30 mL, about 20 mL, about 10 mL, or about 5 mL) of the sterile solution, to provide a final dosage of about 1.2 g of tobramycin.

The above-described method of preparing the inventive pharmaceutical dosage form can consistently and reproducibly produce dosage forms with high dosage accuracy and low variability in the dosage. Moreover, the method is simpler and is significantly less costly than the conventional methods used in the production of tobramycin sulfate powder.

The pharmaceutical dosage form prepared in accordance with the present invention preferably is within about 20% of the label claim. In other words, the amount of tobramycin in the container (as determined by a suitable analytical technique, e.g., HPLC, tobramycin sulfate assay, or the like) preferably is within about 20 wt % of the tobramycin dosage claimed in the product label. Most preferably, the inventive pharmaceutical dosage form has an actual dosage of tobramycin that is within about 10% for its lower end and within about 15% for its higher end of the label claim. By way of example, for 1.2 g dosage vials prepared in accordance with the present invention, with a label claim of 1.2 g of tobramycin, the amount of tobramycin in the vials (e.g., present as tobramycin sulfate), as determined by a suitable analytical technique, preferably is within about 1.08 g to about 1.38 g. Even more preferably, the inventive pharmaceutical dosage form has an actual dosage of tobramycin that is within about 10% of the label claim. Most preferably, the inventive pharmaceutical dosage form has an actual dosage that is within about 5%, about 4%, or about 3% of the label claim.

Also provided by the invention is a method of treating a disease in a patient in need thereof. The method comprises dissolving the inventive pharmaceutical formulation comprising lyophilized tobramycin in free-flowing powder form in a pharmaceutically acceptable solvent to produce a pharmaceutically acceptable solution, and administering the solution to the patient. The lyophilized tobramycin formulation can be administered to a patient in need thereof (e.g., to treat microbial infections) using standard therapeutic methods for delivering tobramycin. While any suitable means of administering the pharmaceutical formulation to a human can be used within the context of the invention, typically and preferably the inventive method of treating a disease in a patient involves administering the pharmaceutical formulation to a human via injection. By the term “injection,” it is meant that the composition is forcefully introduced into a target tissue of the human. The pharmaceutical formulation can be administered to the human by any suitable route, but preferably is administered to the human intravenously or intramuscularly. When the inventive composition is administered by injecting, any suitable injection device can be used. While less preferred, other routes of administration can be used to deliver the pharmaceutical formulation to the human in accordance with the inventive method. Indeed, although more than one route can be used to administer the inventive formulation, a particular route can provide a more immediate and more effective reaction than another route.

The inventive pharmaceutical formulation comprising lyophilized tobramycin can be reconstituted for parenteral administration to a patient using any pharmaceutically acceptable diluent. Preferably, the diluent is Sterile Water for Injection, USP. Alternatively, the diluent may be, for example, 5% or 10% Dextrose in water, USP, 0.9% Sodium Chloride Injection, USP, 5% Dextrose and 0.9% Sodium Chloride, Ringer's Solution, or Lactated Ringer's Injection, USP. Preferably the reconstitution is done in Sterile Water for Injection, USP, and the reconstituted solution optionally is further diluted, e.g., in 5% Dextrose in water, USP, or 0.9% Sodium Chloride Injection, USP.

Any quantity of diluent may be used to reconstitute the lyophilized tobramycin such that a suitable solution for injection is prepared. Accordingly, the quantity of diluent must be sufficient to dissolve the lyophilized tobramycin. Typically, about 50 mL or less (e.g., about 40 mL, about 30 mL, about 20 mL, about 10 mL, or about 5 mL) of diluent are used to reconstitute the lyophilized tobramycin to yield a final concentration of about 40 mg/mL or less (e.g., about 40 mg/mL, about 35 mg/mL, about 30 mg/mL, about 25 mg/mL, about 20 mg/mL, about 15 mg/mL, or 10 mg/mL, about 5 mg/mL, or about 1 mg/mL). More preferably, the reconstituted solution is further diluted to a final concentration of tobramycin of about 2 mg/mL or less (e.g., about 1.5 mg/mL, about 1.0 mg/mL, about 0.5 mg/mL, about 0.4 mg/mL, about 0.3 mg/mL, or about 0.2 mg/mL). Most preferably, the final concentration of tobramycin is from about 0.2 mg/mL to about 2 mg/mL.

Prior to reconstitution, the inventive lyophilized tobramycin dosage form should be stored at controlled room temperature, preferably about 59° F. to about 86° F. (15° C. to 30° C.). Reconstituted solutions of lyophilized tobramycin should be administered to a patient promptly upon constitution. Alternatively, reconstituted solutions should be refrigerated and used within 96 hours. If kept at room temperature, reconstituted solutions of the pharmaceutical formulation should be used within about 24 hours.

Solutions of tobramycin may be further diluted after reconstitution using any suitable diluent. Preferred for further dilution are 5% Dextrose in water, USP, or 0.9% Sodium Chloride Injection, USP. Other fluids for further dilution of solutions of reconstituted lyophilized tobramycin include, for example, 10% Dextrose in water, USP, 5% Dextrose and 0.9% Sodium Chloride, Ringer's Solution, Lactated Ringer's Injection, USP, or Sterile Water for Injection, USP.

The inventive pharmaceutical formulation can be administered to a patient (e.g., a human patient) to treat or prevent any disease or condition against which tobramycin (e.g., tobramycin sulfate) is active. In this regard, the pharmaceutical formulation can be administered to a patient suffering from, for example, septicemia, complicated and recurrent urinary tract infections, lower respiratory infections, serious skin and soft tissue infections including burns and peritonitis, and central nervous system (CNS) infections caused by organisms resistant to other antibiotics.

In addition or alternatively, the inventive pharmaceutical composition can be administered to a patient that has been infected by a microorganism that is sensitive to tobramycin. Such microorganisms include, for example, P. aeruginosa, Proteus sp. (indole-positive and indole-negative), including P. mirabilis, M. morganii, P. rettgeri, and P. vulgaris, E. coli, Klebsiella-Enterobacter-Serratia group, Citrobacter sp., Providencia sp., Staphylococci, including S. aureus (coagulase-positive and coagulase-negative). These microorganisms, however, are merely exemplary. Indeed, the inventive pharmaceutical formulation can be administered to a patient that has been infected with any microoganism that is sensitive to (i.e., whose protein synthesis is irreversably inhibited by) tobramycin.

In addition to the preferred embodiments described herein, the inventive pharmaceutical formulation can comprise additional therapeutic or biologically active agents. For example, therapeutic factors useful in the treatment of a particular indication (e.g., septicemia) can be present. Factors that control inflammation, such as ibuprofen or steroids, can be part of the composition to reduce swelling and inflammation associated with in vivo administration of the composition and physiological distress. Immune enhancers can be included in the composition to up regulate the body's natural defenses against disease. Vitamins and minerals, antioxidants, and micronutrients can be co-administered with the composition.

The following examples further illustrate the invention but, of course, should not be construed as in any way limiting its scope.

### EXAMPLE 1

This example demonstrates the preparation of a liquid composition comprising tobramycin sulfate and a solvent containing tert-butyl alcohol.

A quantity of sterile water for injection (“WFI”) was collected (approximately 80% of total batch quantity) and was placed into a clean, jacketed Type 316 stainless steel compounding tank. The WFI was at a temperature of about 15° C. to 30° C. Tobramycin sulfate, USP raw material (obtained from a commercial supplier, 5.57 kg) was added to the WFI in the compounding tank. The resulting mixture was stirred at 716 rpm until the tobramycin sulfate was completely dissolved in the WFI (at least 10 minutes).

After the tobramycin was dissolved, the solution was stirred at 625 rpm, and 1.59 kg of tert-butyl alcohol, which had been warmed to a temperature of about 30° C. and 45° C., was added during stirring. The mixture was stirred at 719 rpm until the tert-butyl alcohol was completely dissolved (at least 10 minutes).

After the tert-butyl alcohol was dissolved, the batch volume was raised to 45 L (total batch quantity) by the addition of WFI, USP, and the solution was stirred at 714 rpm for at least an additional 10 minutes.

Prior to filtration, an in-process sample was taken from the batch tank, and was subjected to an appearance test (visual examination) and tobramycin sulfate assay. After completion of the in-process appearance test and tobramycin sulfate assay, the cooled solution of tobramycin sulfate in WFI was pumped through a 0.45 μm pre-filter (Opticap® or Durapore® PVDF available from Millipore Corporation) into a filling room using Tygon® tubing. The pre-filter was rinsed with WFI, USP prior to filtration of the tobramycin sulfate solution. Near the end of the filtration step, a sample was collected and subjected to a pre-filtration bioburden test to determine the action limit of the material, which should be not more than 10 CFU/mL.

Following pre-filtration, the tobramycin sulfate solution was passed through a sterile 0.22 μm final filter (Opticap® or Millidisk® PVDF available from Millipore Corporation) and the filtrate was delivered into a clean, sterile receiving carboy using silicone tubing. In a class 100 clean room environment, sterile vials were aseptically filled with the solution and partially stoppered.

### EXAMPLE 2

This example demonstrates a method of lyophilizing a pharmaceutical formulation comprising tobramycin sulfate.

A nitrogen supply was connected to a sterilizing filter assembly on a lyophilizer and the lyophilizer chamber and condenser were steam sterilized for at least 30 minutes using standard sterilization procedures. The minimum chamber drain temperature and minimum condenser drain temperature were each at least 121.0° C. After sterilization, a vessel integrity test was performed. The shelves and condenser plates of the lyophilizer were chilled to −30° C. and −50° C., respectively, and the vessel was leak tested.

The lyophilization process was initiated by pre-chilling the shelves of the lyophilizer. The shelf temperature controller was adjusted to a set point of 5° C. After the pre-chill set point was reached, the shelves were loaded with vials containing a tobramycin sulfate composition prepared as described in Example 1 over approximately 8 hours. After the product solution was completely loaded, the chamber door was closed and the shelf temperature was maintained at the pre-chill set point for at least 60 minutes. The shelf temperature controller was then adjusted to a set point of −50° C. with a ramp time of 265 minutes and the shelf temperature was held at the set point temperature of −50° C. for at least 15 hours.

When the condenser temperature reached −48° C., the primary drying step was performed. The vacuum controller set point was set to 100 μm Hg (13 Pa) and the vacuum alarm high set point was set to 125 μm (16 Pa). Nitrogen gas was used to regulate the pressure. The shelf temperature controller was adjusted to a primary drying set point of −15° C. with a ramp time of 5.5 hours and the shelf temperature was held at the drying set point temperature of −15° C. for 67 hours.

The shelf temperature controller was adjusted to a secondary drying set point of 30° C. with a ramp time of 225 minutes and the shelf temperature was held at the secondary drying set point temperature of 30° C. for at least 18 hours. At the end of the 18-hour secondary drying hold time, the chamber was isolated and the vacuum was released. The pressure was raised to atmospheric pressure by addition of sterile nitrogen gas.

The vials containing the lyophilized tobramycin sulfate were then stoppered with Stelmi 6720GC Gray Lyo stoppers using an internal stoppering mechanism. At the completion of the stoppering step, the chamber was again isolated and the vacuum released. The pressure was raised to atmospheric pressure by addition of sterile nitrogen gas. The lyophilizer chamber was unloaded and the stoppered vials containing lyophilized product were conveyed to a capping machine and sealed with aluminum seals. The vials were then inspected, labeled, and packaged.

The final product was a sterile, white to off-white solid having greater than 98% purity and was suitable for administration by injection. Solutions prepared by dissolving the final product in Sterile Water for Injection were clear and free of particulates.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of producing a stable, sterile pharmaceutical formulation comprising lyophilized tobramycin, which method comprises:
(a) preparing a liquid composition comprising tobramycin and a solvent comprising about 4.5% by volume or less of tert-butyl alcohol,
(b) freezing the composition to a temperature of from about −10° C. to about −70° C., to produce a frozen mixture, wherein the temperature is maintained for from at least about 1 hour to about 30 hours,
(c) subjecting the frozen mixture to a primary drying stage, which comprises applying a vacuum to reduce the pressure to about 100 micron Hg, and, while applying the vacuum, changing the temperature of the frozen mixture to a primary drying temperature of from about −15° C. to about 20° C., wherein the temperature of the frozen mixture in the primary drying stage is changed at a rate of from about 0.1° C. to about 0.5° C. per minute, and wherein the primary drying temperature is maintained for at least about 40 hours to about 80 hours, to produce a first intermediate, and
(d) subjecting the first intermediate to a secondary drying stage, which comprises applying a vacuum to reduce the pressure to about 100 micron Hg, and, while applying the vacuum, changing the temperature of the frozen mixture to a secondary drying temperature of from about 30° C. to about 45° C., wherein the temperature of the frozen mixture in the secondary drying stage is changed at a rate of from about 0.1° C. to about 0.5° C. per minute, and wherein the secondary drying temperature is maintained for at least about 5 hours to about 30 hours, to produce the pharmaceutical formulation, wherein the lyophilized tobramycin is in the form of a free-flowing powder.

2. The method of claim 1, wherein the composition is frozen to a temperature of from about −45° C. to about −60° C.

3. The method of claim 1, wherein the composition is frozen to a temperature of about −45° C.

4. The method of claim 1, wherein the primary drying temperature is from about −15° C. to about 10° C.

5. The method of claim 1, wherein the temperature at which the composition is frozen is held for at least about 10 hours to about 20 hours.

6. The method of claim 1, wherein the temperature of the frozen mixture in the primary drying stage is raised at a rate of about 0.1° C. per minute.

7. The method of claim 1, wherein the primary drying temperature in the primary drying stage is maintained for at least about 60 hours to about 70 hours.

8. The method of claim 1, wherein the primary drying temperature in the primary drying stage is maintained until the temperature of the frozen mixture is about equal to the primary drying temperature.

9. The method of claim 1, wherein the secondary drying temperature is from about 30° C. to about 40° C.

10. The method of claim 1, wherein the temperature of the frozen mixture in the secondary drying stage is raised at a rate of about 0.2° C. per minute.

11. The method of claim 1, wherein the secondary drying temperature in the secondary drying stage is maintained for at least about 10 hours to about 20 hours.

12. The method of claim 1, wherein the secondary drying temperature in the secondary drying stage is held until the moisture content is about 1.0 wt % of the formulation.

13. The method of claim 1, wherein (a) the primary drying temperature is from about −15° C. to about −10° C., (b) the temperature of the frozen mixture in the primary drying stage is raised at a rate of about 0.1° C. per minute, and (c) the primary drying temperature is maintained for at least about 60 hours to about 70 hours.

14. The method of claim 1, wherein (a) the composition is frozen to a temperature of from about −45° C. to about −60° C., and the temperature at which the composition is frozen is maintained for at least about 10 hours to 20 hours, (b) the primary drying temperature is from about −15° C. to about −10° C., and the primary drying temperature is maintained for at least about 60 hours to about 70 hours, and (c) the secondary drying temperature is from about 30° C. to about 40° C., and the secondary drying temperature is maintained for at least about 10 hours to about 20 hours.

15. The method of claim 1, wherein (a) the composition is frozen to a temperature of about −48° C., and the temperature at which the composition is frozen is maintained for at least about 15 hours, (b) the primary drying temperature is about −15° C., and the primary drying temperature is maintained for at least about 67 hours, and (c) the secondary drying temperature is about 30° C., and the secondary drying temperature is maintained for at least about 15 hours.
